(19) United States
(12) Patent Application Publication
(10) Pub. No.: US 2008/0261247 A1
(43) Pub. Date: Oct. 23, 2008

(54) Method of detecting red cell antigen-antibody reactions

(76) Inventor: Parviz Lalezari, Scarsdale, NY (US)

Correspondence Address: HEDMAN & COSTIGAN P.C., 1185 AVENUE OF THE AMERICAS, NEW YORK, NY, 10036, US

(21) Appl. No.: 12/080,507

(22) Filed: Apr. 3, 2008

Related U.S. Application Data
(60) Provisional application No. 60/921,610, filed on Apr. 3, 2007.

(51) Int. Cl. G01N 33/569

(52) U.S. Cl. 435/7.25

(57) ABSTRACT

A process for the detection of antibodies in a test sample by carrying out the following steps:
(a) preparing an essentially isotonic and low ionic strength suspension comprising said test sample and erythrocytes;
(b) incubating the erythrocytes, test sample and low ionic strength medium at 37-45° C. for various time periods to optimize antibody uptake;
(c) combining said suspension with an amount of a solution of hexadimethrine bromide which is effective for agglutination of said erythrocytes and
(d) separating the resultant agglutinates of polymer and erythrocytes from said suspension;
(e) neutralizing the effect of hexadimethrine bromide by adding an effective amount of a gylcosaminoglycan in combination with other polybrene neutralizing agents;
(f) monitoring the resuspended agglutinates for the presence or absence of antibody;
(g) stabilizing the antibody dependent aggregate by adding an effective macromolecule as a component of the dispersing solution or separately and optionally.
(h) packing and reincubating the test red cells to further increase the test sensitivity.

This application claims the benefit of provisional application Ser. No. 60/921,610

### BACKGROUND OF THE INVENTION

Numerous techniques for the detection of antibodies exist. These are utilized in many applications to determine the presence of any, or given, antibodies and to measure their concentration in a variety of fluids, most particularly blood. These techniques are particularly useful for typing erythrocytes and in the cross-matching of blood for transfusion.

The Manual Polybrene Test (MPT) is a rapid, simple and economical test for detection of many red cell antigen-antibody reactions. The processes involved can be divided into three phases, Namely the Sensitization Phase in which the antigen-antibody reaction takes place in a low ionic medium(LIM); the Polybrene-induced aggregation phase in which the sensitized erythrocytes are non-specifically aggregated by Polybrene, a positively charged synthetic polymer, and the Polybrene-Neutralization Phase in which a salt such as trisodium citrate is used to resuspend the aggregates produced by polybrene. The presence of an antibody is recognized by persistence of agglutination after the Polybrene effects have been reversed. Although the MPT is more sensitive than other existing tests for detection of many erythrocyte antibodies, it has been found to lack adequate sensitivity for Kell-related antibodies, a deficiency that has prevented MPT from becoming a universal blood bank test in the countries where Kell-incompatibility is relatively common. This invention describes a new, Polybrene-based test in which the causes of the failure of the original test have been identified and corrected, and higher sensitivity for Kell-related antibodies and many other antigen-antibody reactions is provided. Four innovations have been introduced in the new test: Antibody uptake by the red cells has been enhanced by increasing the time and the temperature of the incubation at LIM sensitization phase, using a new LIM formulation. New Polybrene neutralization solutions have been developed in which the salt concentration has been markedly reduced. High salt concentration was identified as a major cause of break down of antibody-dependent agglutinates at the Polybrene neutralization phase; and at the end of the test, new macromolecules were added to stabilize the antibody-dependent agglutinates. Instability of antibody-dependent agglutinates was identified as another major reason for the loss of test sensitivity, especially for the Kell system.

An optional fourth phase was introduced in the test which involved re-centrifugation of the test tubes and re-incubation of the packed cells for up to 10 minutes and reevaluation of the status of the red cell aggregation. The addition of the fourth phase markedly increased the test sensitivity, especially for the Kell-related antibodies.

The most commonly employed prior art procedures are based on the use of antiglobulin reagent and the manual polybrene test described in U.S. Pat. No. 4,436,825. The antiglobulin procedure is described in an article by Coombs et al at Vol. 26, page 255 of the Brit. J. Exp. Path (1945). These procedures, however, have the drawbacks of being time-consuming and of inadequate sensitivity.

Another method is an automated method described in an article in Transfusion, Vol. 8, No. 6 November-December 1968 by P. Lalezari. That method utilizes another approach, however, that method has substantial drawbacks. It involves careful attention to proportions of ingredients and reagents as well as complicated equipment. Further, it too is time-consuming.

The disclosures of these articles are incorporated herein by reference. The drawbacks of these existing techniques pose substantial impediments to the need for fast and simple detection with acceptable accuracy. The need for improved detection techniques is therefore clear.

### SUMMARY OF THE INVENTION

A process for the detection of antibodies in a test sample comprising serum or plasma:

(a) preparing a suspension of erythrocytes in net negatively-charged form;
(b) mixing the said erythrocyte suspension with the test serum or plasma or their derivatives, as the source of antibodies;
(c) suspending the said erythrocyte-antibody mixtures in a low ionic solution and maintaining the combination from sixty seconds to 10 minutes, preferably for 2-5 minutes at 40 to 45° C., Alternatively, the said suspensions are kept 37° C. for 3 to 10 minutes, preferably for 5 minutes, or at 39° C. for 5 minutes.
(d) combining said suspension with an amount of a solution of hexadimethrine bromide, protamine or polylysine which is effective for agglutination of said erythrocytes;
(e) separating the resultant red cell agglutinates by centrifugation from the LIM said supernatant and,
(f) neutralizing the effect of hexadimethrine bromide, protamine or polylysine by adding an effective amount of a neutralizing solution;
(g) stabilizing the antibody dependent aggregate by adding an effective amount of a macromolecule;
(h) monitoring the status of the erythrocyte agglutination;
(i) optionally, re-centrifuging the test tubes and re-incubating the pellets for 5 to 10 minutes at 37 or 43 degrees centigrade and,
(j) monitoring the status of the erythrocyte aggregation again.

While the steps are described above as comprising the invention, in particular cases these steps may be the only steps that are used and as such they will consist essentially of the steps of the invention in that other steps that have a material effect will be excluded.

The persistence of agglutination indicates the presence of an antibody against the test red cells.

The invention also includes a kit for use in the detection of antibodies, wherein the kit comprises:

(a) a low ionic strength medium having a sensitization effective pH 6.0-7.0;
(b) a solution of a positively charged compound such as hexadimethrine bromide, protamine or polylysine in a concentration which is effective for agglutination of erythrocytes; and
(c) A salt solution of negatively charged macromolecules combined with sodium citrate and similar salts. These combinations are effective to neutralize the effects of hexadimethrine bromide and at the same time stabilize the agglutinates. Alternatively, a solution of a glycosaminoglycan (GAG), combined with other agents, which is effective to neutralize the effect of hexadimethrine bromide.

The preferred GAG has a low molecular weight, i.e. less than about 10,000 daltons, such as dalteparin.

This test produces accurate results which are substantially independent of most variations in the amount or type of ingredients and reactants employed.

### DETAILED DESCRIPTION OF THE INVENTION

The process may be completed in mere minutes with simple laboratory equipment. The present process is suitable for detection of antibodies in any normally aqueous sample. Most commonly, it is applied to a blood serum or plasma (including derivatives thereof). Erythrocytes are added to the sample after an optional washing step with isotonic salt solution. Desirably the sample contains from 0.5 to 3% more preferably 1% to 2%, suspended erythrocytes (or antigen coated erythrocytes) by total volume. These erythrocytes are normally obtained from a different source donor to ensure that they will accept any antibodies present in the sample. In the case of cross-matching blood for transfusion, the red cells are from normal blood donors.

Erythrocytes may be obtained from the same donor as the serum if the presence of an autoantibody is being sought.

In the sample suspension, the erythrocytes should exhibit a net negative charge. This is the normal or native state of an erythrocyte. In this form, they will possess maximum sensitivity to subsequent agglutination. The negative charge is ensured by maintaining a pH of between about 6.0 and 7.0 in 0.5 to 1.5 ml, preferably 11.0 ml of a low ionic strength medium.

Maximum agglutination is also achieved in an essentially isotonic and low ionic strength medium. The low ionic strength medium appears to sensitize the erythrocytes for acceptance of antibodies. Therefore a final salt concentration of from 0.01 to 0.05 molar, especially about 0.03 molar is preferred, although many additives may be employed to achieve LIM suspension conditions, addition of neutral amino acids such as glycine are most convenient for increasing osmotic pressure. Virtually any salt may be used to increase ionic strength and simple dilution will reduce both of these conditions.

In an optional preferred embodiment, a chelating agent is employed in the low ionic strength medium. The chelating agent provides the salt and in addition facilitates the polybrene (hexadimethrine bromide)-neutralization process. If polylysine is used it should be of the formula (Lys)ₙ where n is about 10 to about 2,000.

A preferred low ionic strength medium comprises 1.5-2.0 w/v % of glycine; 0.01 to 0.4 w/v % of di-sodium EDTA and 0.25-1 w/v %, preferably 0.5 w/v % of polyvinylpyrrolidone (PVP, weight average mol. wt. of 90,000-1.3 million, preferably 360,000-1.3 million. PVP has the following properties: (a) like the sodium salts of polycarboxylic acids, PVP has the ability to act like an antiglobulin reagent and will directly aggregate antibody coated red cells: (b) PVP is compatible with Polybrene and in fact enhances Polybrene's red cell aggregating activity: (c) PVP does not contain salts and therefor it does not alter the nature of the low ionic strength medium; (d) as a polymer, PVP increases the viscosity of the test medium and helps to stabilize the antibody-dependent red cell aggregates after the Polybrene effect has been neutralized in the last phase of the reaction.

The addition of a chelating agent, such as di-sodium EDTA, to the low ionic strength medium has been shown to facilitate the dispersion of non-specific aggregates in the third phase of the reaction. In a typical test, 0.1 ml of test serum or plasma is mixed with 0.05 ml of the red cell suspension and one ml of the low ionic strength medium and incubated as described below.

An optional low ionic medium for combination with the test sample therefore comprises an aqueous solution of a chelating agent and glycine or 0.24 molar solution of other neutral amino acids as described above without additional PVP. The chelating agent may comprise di-sodium EDTA or similar molecules.

### The Time and Temperature of the Sensitization Phase.

The mixture of the red cell suspension, the test antibody and low ionic strength medium can be incubated at various temperatures, i.e. 37-43° C. based on the nature of the specificity being tested, as described above.

In addition, the Polybrene-induced Red Cell Aggregation Phase may be modified by increasing the Polybrene concentration from 0.05% to 0.2% w/v. After the addition of Polybrene, the test tubes are centrifuged for 15 seconds at 1000×g, the cell free supernatants are decanted, and the pellets are gently agitated to free them from the glass.

The Polybrene neutralization phase may be modified by using a combination of the negatively charged macromolecules with salts for effective Polybrene neutralization. This combination offers the following advantages: (a) the macromolecules used are selected on the basis of their ability to react as an antiglobulin reagent and thus enhance the sensitivity of the original Polybrene test; (b) by their Polybrene-neutralizing properties (due to their negative charge) help dispersion of non-specific aggregates, and (c) by high viscosity, serve as an stabilizer of the antibody-dependent aggregates. Combining the negatively charged macromolecules with salts allows to reduce the salt concentration which has been shown to break down the antibody-dependent aggregates if used in high concentrations. The negatively charged macromolecules identified to be useful for this purpose include the following: Sodium salts of Polyacrylic acids at various weight average molecular weights ranging from 15,000 to 3 million Dalton. An example that has been found to be effective is Polyacrylic acid sodium salt with an weight average molecular size of 240,000 Dalton. An example of an effective Polybrene-neutralizing solution is Polyacrylic acid sodium salt at 0.5% combined with trisodium citrate at 0.1 molar concentration and dextrose at 2.5%. Other positively charged macromolecules found to be useful include various Polycarboxylic acid salts and Poly[(isobutylene-alt-maleic acid)NH₄-co-(isobutylene—alt-maleic anhydride)] (PIMA), In the above Polybrene neutralizing solutions, the trisodium salt can be replaced by effective concentrations of sodium or ammonium salts of mono-, di-, and tricarboxylic acids such as acetate, oxalate, malate, suberate, citrate and the like. If the test results do not show detection of any antibody, a fourth phase may be added by re-centrifuging the test tubes for 10 seconds, followed by re-incubation for 5-10 minutes at 37-43° C. This additional step greatly increases the test sensitivity, especially for the Kell type antibodies.

Once the suspension has been prepared, antibody-erythrocyte sensitization for subsequent steps is rapidly reached under appropriate incubation time and temperature (if, that is, antibody is present in the test sample). In order to further facilitate antibody uptake by the red cells, the suspension need be maintained for 3 to 10 minutes, preferably for 5 minutes, at 37° C., or for 2 to 4 minutes, preferably 2.5 minutes at 43° C., or a temperature of 39° C. for 4 minutes before addition of polymer.

Agglutination of the suspended erythrocytes is accomplished by addition of a solution of hexadimethrine bromide and after 15-30 seconds at room temperature, centrifugation for 20 to 60 seconds at 1000×g, which produces a button of aggregated erythrocytes. The cell free supernatant is then decanted and a solution of a negatively charged macromolecule, namely 0.5 w/v % sodium salt of polyacrylic acid (MW 240,000) dissolved in 2.5 W/v % dextrose and 0.1 molar trisodium citrate, or GAG combined with other agents is added to the cell button for Polybrene neutralization.

The GAG's that are useful in the invention include, unmodified heparin, Heparan (heparin mono-sulfate), and low molecular weight heparin (3,000 to 10,000 daltons with a weight average of about 5,000). A preferred GAG is a low molecular weight heparin such as the heparins described in U.S. Pat. No. 4,303,651, which is incorporated by reference. Dalteparin sodium is a commercially available low molecular weight heparin under the Fragmin trademark, or Enoxaparin, under the Lovenox trademark. This heparin is described in the PDR60th Ed (2006) at pages 2638-2642, which is incorporated by reference.

The concentration of the GAG in an aqueous solution may range from 5 to 50 units per ml. The amount of the solution of the GAG that is added to the suspension is not critical and it may range from 0.1 to 0.2 ml. Optionally, the following materials may be added with the GAG solution; tri-sodium citrate, carboxymethylcellulose sodium (low viscosity i.e. 50-200 centipoise 4% solution in water at 25° C.), polyvinylpyrrolidone, Poly[(isobutylene-alt-maleic acid)NH4-co(isobutylene-alt-maleic anhydride)] (PIMA), bovine serum albumin and other similar compounds, in amounts which enhance the neutralizing effect of the GAG.

A combination of some of these agents without GAG may also be effective to neutralize the polybrene effects.

Normally, sufficient neutralizing agents are employed to disperse the non-specific Polybrene-dependent aggregates, using gentle agitation.

Addition of dispersing solutions requires 15 to 30 seconds to completely perform its function. Immediately thereafter, 0.1 to 0.2 ml of a macromolecule solution which is capable of stabilizing the aggregates by raising the viscosity of the medium. A suitable macromolecule may be composed of 10 to 15% polyethyleneglycol, with average molecular weight of 8,000 Daltons, low viscosity carboxymethylcellulose, (PIMA) or 7 to 10% PVP K90, or similar compounds, may be added to the cells and mixed to stabilize the agglutinates, if present. The addition of stabilizers is needed only when GAG or small molecule salts are used for Polybrene neutralization. Stability of the antibody-dependent aggregates is provided by negatively charged macromolecules when are used for Polybrene neutralization.

In the absence of antibodies, the agglutinates rapidly dissociate and reassume a suspension or colloidal form. In contrast, the presence of antibody is evidenced by its coupling effect on the erythrocytes. Therefore antibodies are revealed or detected by persistence of the agglutinates within the dispersion.

These results allow detection to occur through simple monitoring of the dispersed agglutinates. For example, visual monitoring (aided, if desired, by a microscope or a magnifying mirror) allows rapid observation of the degree, if any, to which the agglutinate dissociates. Such dissociation occurs in inverse relationship to the concentration of antibody in the initial test sample.

This relationship also allows for precise quantitative measurement of antibody concentration. By use of either standardized conditions in the process or duplication utilizing control samples, accurate quantitative analysis is achieved.

As described earlier, the process of this invention may be carried out at different time-temperature combinations, offering multiple options. Use of other temperatures may be advantageous to permit separate detection of cold-reactive antibodies. These known antibodies preserve agglutination only at lower than ambient temperatures. Consequently, they may be detected only by chilling the agglutinates to from about 0° C. to 6° C. prior to dispersion and monitoring.

Accordingly, this process also allows qualitative detection of antibodies which are cold versus ambient reactive.

The process of the invention may be adapted for use in automated equipment by using a hexadimethrine bromide solution as a step in the automated analysis according to the procedures described herein.

The process of this invention will be more fully described and better understood from the following examples.

### EXAMPLE I

The test is carried out as follows: 0.05 ml (one drop) of a 3% red cell suspension are mixed with 0.1 ml (two drops)test serum or plasma in one test tube. In another tube, 0.05 ml of the red cells are mixed with 0.1 ml of normal serum or plasma to be used as control.

To each tube then one ml of the Low Ionic Medium (LIM), composed of 0.3 w/v % di-sodium salt of ethylenediaminetetra acetic acid dissolved in 1.8 w/v % Glycine and 0.5 w/v % PVP with 360,000 weight average MW, is added. The cell-serum-LIM mixtures are incubated for 5 minutes, or for up to 30 minutes, at 37° C. to allow antibody uptake by the red cells. Alternatively, they may be incubated at 43° C. for 150 seconds or for 4 minutes at 39° C. to enhance antibody uptake by the red cells. In the next step, 0.1 ml 0.1% hexadimethrine bromide diluted in 0.9 w/v % NaCl solution is added to each tube, and after 15 to 30 seconds at ambient temperature, the tubes are centrifuged for 15 to 60 seconds and the supernates are decanted. When dalteparin sodium, or enoxaparin sodium are used in the test, the hexadimethrine bromide effects are neutralized by adding 0.1 ml of a dispersing solution of 0.6 units of the low molecular heparins in 0.02 molar sodium citrate and 0.5 w/v % PVP K90. The concentrations of the low molecular heparins may vary from 3 to 50 units per ml. If unmodified heparin is used, its concentrations may vary from 10 to 100 units per ml, and the amount of sodium citrate ranges from 0.005 to 0.1 molar. PVP concentration may vary from 0.2 to 2%. The tubes are gently mixed and within 30 seconds 0.1 ml of the stabilizer composed of 15% PEG is added and mixed. An alternative dispersing solution used is composed of 0.5 w/v % polyacrylic acid (weight average MW 240,000 Dalton) in 0.1 molar tri sodium citrate. Several other dispersing solutions have been found to be successful for neutralizing the Polybrene effects. These include a solution composed of 12 units of Deltaparin/ml and 0.2 w/v % PIMA, 0.02 molar trisodium citrate and 0.375 w/v % bovine serum albumin.

Another example is a Polybrene neutralizing solution without any heparin derived material which is composed of 0.25 w/v % low viscosity carboxymethylcellulose with 0.03 molar trisodium citrate and 0.75 w/v % bovine serum albumin.

### EXAMPLE II

A subsequent test was carried out to compare the use of dalterperin sodium as a neutralizing agent for the hexadimethrine bromide with the prior art hexadimethrine bromide method (MPT) and the standard antiglobulin test or AGT. In these experiments, dilutions of three different representative antibodies, i.e., anti-Rh, Kell and Fy were used as well as a non-reactive control. For maximum sensitization, the red cell-antibody mixtures were incubated at 37° C. for 30 minutes before the tests were carried out In the dalteparin test, the sensitized cells were mixed with one ml of glycine-based LIM, Polybrene added and after centrifugation and decantation of the supernates, the aggregates were treated with 0.1 ml of 6 units/ml of dalteparin combined with 0.0025 molar trisodium citrate. For the AGT, the sensitized cells were washed three times before being mixed with an antiglobulin reagent and centrifuged.

The results were as follows:

| Antibody | Dalterperin | MPT | AGT |
|---|---|---|---|
| Anti-Rh | 4+ | 1+ | trace |
| Anti-K | 4+ | 1+ | 2+ |
| Anti-Duffy | 3+ | 1+ | negative |
| Control | negative | negative | negative |

The results indicate that dalteparin test is more sensitive than both AGT and MPT. In the MPT, the weak reactions were unstable, and the antibody agglutinates rapidly dispersed.

In the foregoing Examples, monitoring was performed simply through visual inspection of macroscopic agglutinates. They therefore constituted only negative-positive detection tests. More accurate and quantitative results may be obtained by careful microscopic monitoring involving detection of degree of erythrocyte dissociation. Similar results may be obtained by other techniques including, for example, photometric analysis of the red cell dispersion shortly after addition of dispersion and stabilizing solutions. This allows precise measurement of degree of dissociation.

For these quantitative processes of the present invention, control samples are often run in tandem with the test sample. This ensures accuracy of detection measurement. Such controls may either possess none or a predetermined amount of antibody according to standard analytical procedures.

In addition to the various reagent constituents previously mentioned, others may be present. These constituents include bactericides, such as sodium azide, to avoid contamination; sugars, such as dextrose, to provide nutrients and sufficient osmotic pressure to ensure normalcy of the erythrocytes; and the like for similar known or apparent purposes.

It is to be understood that these changes may be made in the following exemplary embodiments in the light of the above teachings. Additional modifications and/or variations may also be made without departing from the scope and spirit of the invention which therefore shall be measured by the claims which follow.

What is claimed is:

1. A process for the detection of antibodies in a test serum or plasma sample comprising:
(a) combining said test serum or plasma sample with erythrocytes in negatively charged form, mixed in a substantially isotonic and low ionic strength suspension to form an erythrocyte suspension;
(c) combining said erythrocyte suspension with an amount of a solution of positively charged compound which is effective for agglutination of said erythrocytes;
(d) separating the resultant agglutinates of polymer and erythrocytes from said suspension;
(e) neutralizing the effect of said positively charged compound by adding an effective amount of a dispersing agent; and
(f) monitoring the treated cells for the presence or absence of antibody as indicated by persistence or absence of agglutinates.

2. The process of claim 1 wherein the positively charged compound is selected from hexadimethrine bromide, protamine and polylysine.

3. The process of claim 1 including the additional step of re-centrifuging, reincubating and re-examining the test tubes for the presence of agglutinates if no residual agglutinates are found after the first centrifugation and incubating steps which indicate the presence of antibodies.

4. MONTEVEGLIO BOLOGNA. The process of claim 1 wherein the dispersed agglutinates without bound antibody are monitored for dissociation.

5. The process of claim 1 wherein the undispersed agglutinates with bound antibody are monitored.

6. A process for the detection of antibodies as defined in claim 1 wherein in step (b) the low ionic strength medium contains polyvinylpyrrolidone.

7. A process for the detection of antibodies as defined in claim 1 wherein in step (e) the dispersing agent contains sodium salts of polyacrylic acid, low viscosity polycarboxylic acid, PIMA or similar compounds in combination with sodium or ammonium salts of mono-, di- or tri carboxylic acids.

8. A process for the detection of antibodies in a test sample comprising:
(a) preparing an essentially isotonic and low ionic strength suspension comprising said sample and erythrocytes in net negatively-charged form;
(b) maintaining said suspension for a period of time at various temperatures from 60 seconds to 30 minutes.

9. The process of claim 5 wherein the dispersed agglutinates without bound antibody are monitored for dissociation.

10. The process of claim 5 wherein the undispersed agglutinates with bound antibody are monitored.

11. The process of claim 5 wherein the low ionic strength suspension of step (a) further comprises ethylenediamine tetra-acetate and/or polyvinylpyrrolidone.

12. The process of claim 5 wherein the low ionic strength suspension of step (a) further comprises a sensitization effective amount of ethylenediamine tetra-acetate having a pH of from about 6.0 to 7.0, and a 0.24 molar solution of a neutral amino acid.

13. The process of claim 5 wherein the low molecular weight heparin is dalterperin sodium.

14. The process of claim 1 wherein the suspension has a pH of between about 6.0 and 7.0.

15. The process of claim 1 wherein the antigen is native to the erythrocytes.

16. A kit for use in the detection of antibodies comprising (a) a medium having a sensitization effective pH;
(b) a solution of a positively charged compound selected from the group consisting of hexadimethrine bromide, protamine or polylysine in a concentration which is effective for agglutination of erythrocytes; and
(c) a solution of a GAG which is effective to neutralize the effect of hexadimethrine bromide.

17. A kit as defined in claim 16 where the GAG is a low molecular weight heparin.

18. A kit as defined in claim 16 where the solution of the GAG also contains an effective amount of a polyacrylic acid

19. A kit as defined in claim 17 where the low molecular weight heparin is dalterperin sodium.
